(19) United States
(12) Patent Application Publication
(10) Pub. No.: US 2016/0367830 A1
(43) Pub. Date: Dec. 22, 2016

(54) System For Visualizing Treated Skin Temperature

(71) Applicant: Syneron Medical LTD., Yoqneam Illit (IL)

(72) Inventor: Shmulik Eisenmann, Pardes Chana-Karkur (IL)

(21) Appl. No.: 14/745,522

(22) Filed: Jun. 22, 2015

(51) Int. Cl. A61N 1/40; A61N 7/02; A61N 5/06; A61B 5/01; A61B 5/00

(52) U.S. Cl. CPC A61N 1/403; A61B 5/015; A61B 5/0059; A61B 5/742; A61N 5/0616; A61N 5/0625; A61N 7/02; A61N 2007/0034

(57) ABSTRACT

An apparatus is provided for thermal skin treatment including an infrared camera and video camera. The apparatus includes a processor that supports processing of the thermal image, display of the thermal image as a visible image and combination of the thermal image with the image obtained by the video camera.

### TECHNOLOGY FIELD

The present description relates to a system for visualizing a cosmetic treatment of a human or animal skin segment and in particular for visualizing temperature distribution of the treated skin segment.

### BACKGROUND

Adipose tissue is frequently treated non-invasively by different energies coupled to the skin. Typical types of energies that may be found in use for skin treatment include ultra sound (US) energy, Radio Frequency (RF) energy, microwave (MW) radiation or radiation energy emitted by a source of light or heat. The skin treatment energy is coupled to the skin by an applicator. The size of the applicator defines to some extent the segment of skin or tissue to which the skin treatment energy is transferred. In order to treat other skin segments, the applicator is repositioned or re-aligned across a large segment of the skin and activated to couple treatment energy to this segment of skin.

Different types of energy are frequently used for circumference reduction, adipose tissue removal, and other cosmetic procedures where application of skin treatment energy could bring a desired beneficial treatment effect. It is known that in order to get a successful skin treatment the temperature of the treated skin area should be maintained at a certain temperature for example 40 degrees Celsius or higher for a certain time for example 30 or 40 minutes and to have it evenly distributed on the treated area. At the time the applicator is repositioned to treat other skin segments, the temperature of the earlier treated segments of skin cools down reducing the effectiveness of the treatment.

Reliance on the caregiver expertise for repositioning of the applicator frequently causes some of the skin treated areas to receiving a lower than desired portion of energy and be at a temperature lower than the optimal treatment temperature, while other skin areas could receive a larger than desired portion of energy and be at a temperature higher than the optimal treatment temperature. The need to accurately identify the temperature readings or representation across the treated skin area under such conditions may represent a serious challenge to any caregiver, rendering the skin treatment dependent on the caregiver's expertise.

### GLOSSARY

The term “skin” as used in the present disclosure includes the outer skin layers such as stratum corneum, dermis, epidermis, connective tissue and the deeper subcutaneous layers such as adipose tissue. The terms “tissue” or “skin” as used in the present disclosure have the same meaning and are used interchangeable through the text of the disclosure.

The term “skin treatment energy” as used in the present disclosure means any one of energies facilitating achievement of a desired skin treatment effect. Such energy could be a mechanical energy, a thermal energy, optical energy, electrical energy, electromagnetic energy and a mix of all or some of them.

The term “energy to skin applying element” as used in the present disclosure means an element, for example, an applicator, operative to receive skin treatment energy from a source of such energy and couple or apply the received energy to a treated segment of skin. An electrode applying RF energy to skin, an ultrasound transducer, a mechanical element, a source of light, a microwave antenna could be such elements.

The term “temperature mapping” as used in the present disclosure means an image processing method to generate a real-time map of the skin or tissue temperature.

The term “tissue or skin affecting energy” as used in the present disclosure means energy capable of causing a change in the tissue, or supporting such change. Such energy for example, may be RF energy, optical radiation in visible or invisible part of electromagnetic spectrum, ultrasound waves energy and MW energy.

The term “computer” as used in the present disclosure means a device including a processing unit capable of receiving data or information, processing it, and delivering the data processing results to another device. As such, a computer may include, as non-limiting examples, a personal computer, a PDA computer, a mobile telephone, a RT controller and similar devices. Typically, a computer as defined herein would have a display or communicate with a display. The display could be a touch type of display such that the caregiver could use the display to enter commands or a monitor screen that displays information and images.

The term “video” or video camera as used in the present disclosure is related to a device configured to capture the visual presentation of the treated skin area and present it on a display screen.

### SUMMARY

A skin by heat treatment monitoring system that supports continuous or periodic monitoring and visualization of the temperature distribution of the treated skin segment. The system provides a temperature distribution map that could be obtained by an IR (infrared) imager or camera and may be displayed to the caregiver during the thermal treatment. Infrared imaging techniques reveal the temperature distribution resulting from the application of skin treatment energy. The temperature distribution map is combined into a single image with a visible image of the treated skin segment generated by one or more video cameras that provide a visible image of the portion of the skin segment and skin segments surrounding the treated skin segment. The combined image facilitates proper location and repositioning of the applicator.

### BRIEF DESCRIPTION OF THE DRAWINGS

FIG. 1 is an example of known skin treatment procedure;

FIG. 2 is an illustration of the present system for skin treatment;

FIG. 3 is an example of a thermal or temperature map; and

FIG. 4 is an example of a combined temperature and location representation.

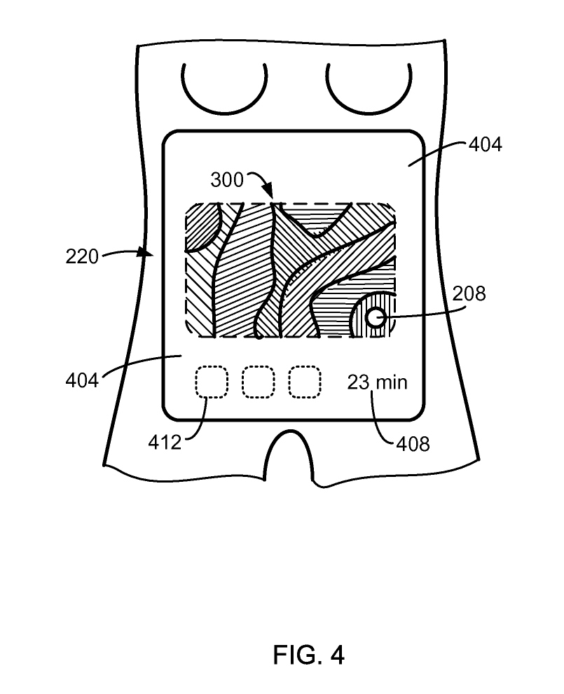

### DETAILED DESCRIPTION OF AN EXEMPLARY EMBODIMENT

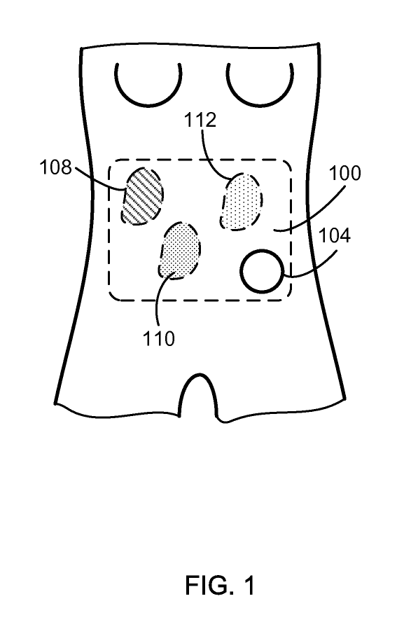

Reference is made to FIG. 1, which is an example of a known skin treatment procedure. The Figure illustrates application of skin treatment energy to abdomen 100 of a person. A thermal energy to skin applying element such as an applicator 104 is used to treat abdomen 100 of a person that desires to reduce for example, abdomen circumference. Segments 108-112 have been treated at the same treatment session a couple of minutes or tens of minutes earlier and then applicator 104 was repositioned to another skin segment.

The current application suggests a process of continuous monitoring of the earlier treated and currently treated skin segments. An infrared camera images the treatment area or a larger area than the treatment area and provides the information collected to a control computer including a processor. The processor processes the thermal image of the treated skin segment and provides a visual feedback to the caregiver. Based on the feedback the caregiver could decide where to position the thermal energy to skin applying element (applicator 104) to maintain the treated skin temperature relatively constant for a desired time.

The skin temperature distribution data obtained by an IR (infrared) imager or camera could be displayed to the caregiver like a thermal or skin temperature distribution map of the treated skin segment and/or used for feedback control for the thermal skin treatment. Infrared imaging and processing technique reveals the temperature variations resulting from the operation and repositioning of the applicator. Such skin temperature distribution map revealing technique supports visualization of a larger skin area than thermistors or thermo-couples support.

The method and apparatus facilitate navigation of the applicator according to the thermal map of the skin or skin temperature distribution map, which improves the quality of the skin treatment and makes the results of the skin treatment more predictable. Temperature on the skin is indicative of the temperature of the adipose tissue or fat located below the particular skin surface area. Based on the measured on the surface of skin temperature and the irradiation time one can calculate the thermal dose received by a particular fat volume of segment.

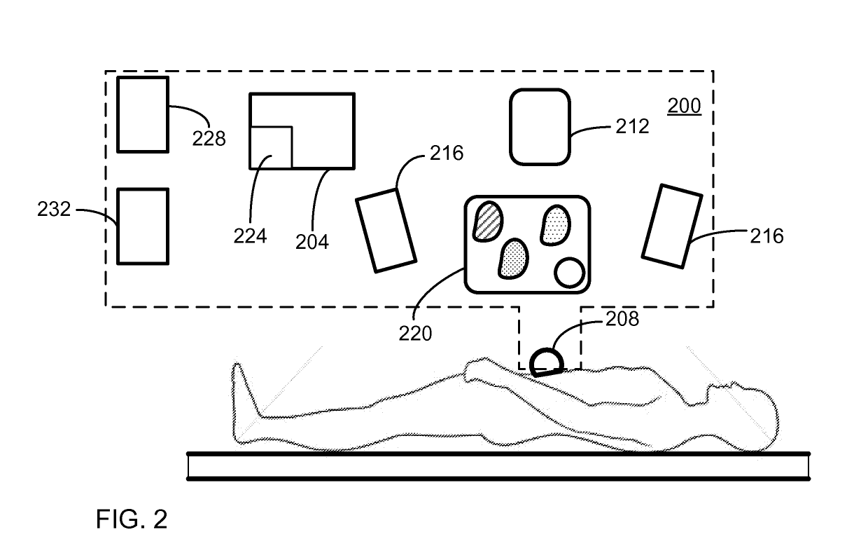

FIG. 2 is an illustration of the present system for skin treatment. System 200 includes a control unit 204, an applicator 208, an infrared imaging device such as infrared imager 212, a number of video cameras 216 and a display 220. Infrared imager 212 is configured to detect infrared radiation from at least one skin segment and to provide a signal reflective of a temperature distribution of the skin segment. The signal reflects the temperature emitted by a treated skin segment and provides a thermal image or temperature distribution of the treated by the skin treatment energy skin segment to control unit 204 and in particular to a processor 224. The processor could be such as a computer or any other device consisting of hardware, firmware with processing capabilities. Processor 224 is configured to receive the signal that reflects the temperature emitted by a treated skin segment and to determine at least the temperature of at least a portion of the skin segment. The processor 224 is also configured to receive data relating to a location of applicator 208, which is configured to apply treatment energy to the treated segment of the skin. Processor 224 is further configured to output a display signal to display 220, the display signal being reflective of the thermal image or temperature distribution of a treated skin segment and the location of the applicator.

System 200 could further include one or more sources 232 of skin treatment energy and optionally sources of negative/vacuum pressure, which could be vacuum pumps 228. The skin treatment energy could be anyone of a group of treatment energies consisting of RF energy, ultrasound energy, optical radiation energy, microwave energy or a combination thereof. Respective sources 232 of the treatment energies could be incorporated into control unit 204 or into a standalone packaging. Treatment energy sources 232 provide treatment energy sufficient to heat the treated skin segment to a temperature of 40 degrees Celsius or higher temperature.

In some examples a “probe” low power pulse of skin treatment energy could be applied to the skin. IR imager 212 or a simple thermal sensor could be used to measure temperature of the skin area affected by the skin treatment energy “probe” pulse. Based on the measured skin temperature processor 224 could calculate most suitable skin treatment energy parameters.

Control unit 204 and in particular processor 224 could be configured to govern operation of the IR imager 212 and video cameras 216. Control unit 204 and in particular processor 224 could be configured to receive the IR image from the IR imager, process it and display a color or grey scale image of the processed image on display 220.

Infrared imager 212 could be almost any infrared camera supporting temperature sensitivity of 0.5 K or better. Camera 212 could have a field of view sufficient to support imaging of an area of the treated skin segment with dimensions of 50×50 cm or smaller.

Imager or infrared camera 212 could be equipped by an optical zoom system supporting imaging of a large area or segment of the treated skin or a small area of the treated skin segment.

Processor 224 is configured to receive the thermal image or temperature distribution of the treated by the skin treatment energy skin segment and determine the temperature of the treated skin segment. Video cameras 216 that could have partially or fully overlapping field of view, also communicate the captured images to processor 224, which based on the video images is configured to determine the location of applicator 208 configured to apply to the skin different types of skin treatment energy. Processor 224 based on the signals received from the infrared imager 212 continuously or at predetermined intervals updates the displayed thermal image of the treated skin segment and based on the signals received from video cameras 216 updates the location of applicator 208 on the display 220. Based on analyses of the thermal image or temperature distribution of the treated skin segment, processor 224 may also configured to generate a skin treatment protocol.

Processor 224 may combine the temperature distribution map into a single image with a visible image of the treated skin segment generated by one or more video cameras that provide a visible image of the portion of the skin segment and skin segments surrounding the treated skin segment. The combined image facilitates proper location and repositioning of the applicator.

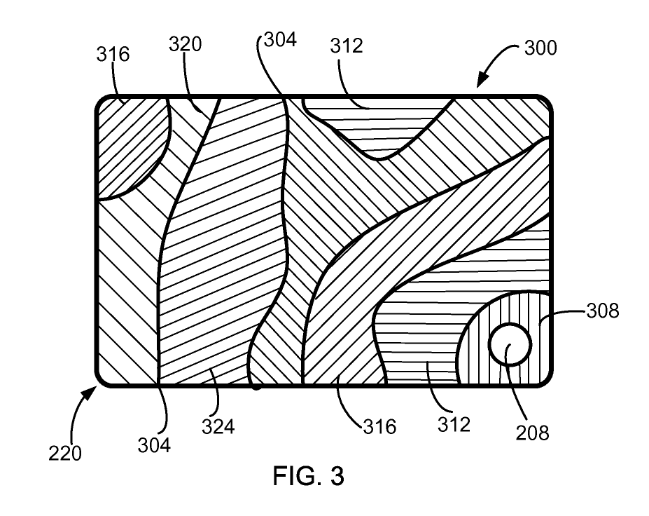

Display 220 is configured to receive from processor 224 the combined thermal image of the treated skin segment and the location of the applicator 208, within the treated by the skin treatment energy skin segment, and display the temperature of the skin segment or thermal map 300 (FIG. 3) of the treated by the skin treatment energy skin segment and applicator 208 location on the same image. The image provided by video cameras 216 could include skin areas in the environment (surrounding) the skin treatment area. Display 220 is also configured to receive from processor 224 only the thermal image of the treated skin segment and the location of the applicator 208, within the treated by the skin treatment energy skin segment,

FIG. 3 is an example of such thermal map or temperature or temperature distribution map of the treated skin segment image. Although, for the clarity of explanation lines 304 separate between skin segments with different temperature, the transition between different temperature areas of the treated skin segment is smooth. Based on the displayed thermal map or temperature distribution map of the treated skin area and location of applicator 208 the caregiver could decide where and when to reposition applicator 208 to maintain the treated skin segment temperature to receive a uniform/homogenous treated skin segment temperature distribution that will result in a proper skin treatment effect. Such temperature could be 40 degrees Celsius or higher. The method is not limited to a specific temperature range and could be applied to skin treatments that require a different temperature range.

The thermal map or temperature distribution data obtained by infrared camera 212 may be displayed to the caregiver and/or used for feedback control of the treated skin segment. The caregiver could for example, more frequent reposition applicator 208 to faster cooling skin areas of the treated skin segment. Furthermore, the processor 224 could be set to issue a warning when a temperature of an area of the treated skin segment exceeds the value pre-determined by a threshold set the caregiver or the processor before the thermal skin treatment. In a similar way processor 224 could alert the caregiver when the temperature of a particular skin area falls below a temperature value considered too low to affect the skin treatment and cause a desired skin effect.

Display 220 in addition to IR image could display images 404 captured by the video cameras 216, the treatment process progress indicator 408 and other treatment process parameters. Display 220 could include a number of soft keys 412 to set the skin treatment protocol. Alternatively, a keypad or a keyboard could be used to set the skin treatment protocol. Although the control unit 204 and other modules of system 200 are shown as separate modules, they are connected by means of cables (not shown) or by wireless communication facilities such as for example Bluetooth®. It will be appreciated that in some examples, the control functions performed by computer 204 could be distributed between different modules of thermal imaging system 200.

The processing unit of thermal imaging system 200 is configured to receive data from the IR camera in a continuous mode or at a different/plurality of intervals. The frequency of the intervals could change and be different before the energy is applied to the skin segment, concurrently with the application of energy to the treated skin segment and after application of the skin treatment energy. Data processing unit 224 is configured to process the received thermal image and construct a thermal/temperature map of the skin segment being treated. According to the mode of operation the thermal map may be continuously updated or according to the frequency of IR camera 212 image sampling intervals.

Availability of a thermal map of the treated skin segment supports changes or adaptation of the thermal treatment protocol to the specific treated person or generation of a new thermal treatment protocol to be used in repeat treatments.

Alternatively, the temperature map of the treated area could be analyzed by processor 224 or an additional computer that would issue and check the desired applicator 208 position on the treated skin segment and issue recommendation on how and where to reposition applicator 208.

System 200 significantly reduces and almost eliminates influence of the caregiver expertise on the skin by heat treatment process. System 200 supports a method for visually presenting a thermal image of a treated skin segment. The method includes use of a treatment energy providing element or applicator 104 to apply at least one skin treatment energy to a segment of skin and treating by skin treatment energy the segment of skin.

Use of an infrared imager 212 configured to capture a thermal image of a treated skin segment and provide a signal reflective of temperature of the treated skin segment supports decision for adaptation or modification of a particular thermal skin treatment. Communication of the thermal image of the treated skin segment to a processor, configured to process the thermal image and provide the thermal image to a display configured to display a thermal image reflective of the treated skin segment temperature and location of the treatment energy providing element visualizes the skin thermal image or temperature distribution and supports repositioning of the treatment energy providing element such as to receive a uniform/homogenous treated skin segment temperature.

Use of the thermal image reflective of temperature distribution across the treated segment of the skin supports decision for adaptation or modification of a particular thermal skin treatment. Employing one or more video cameras 216 to image the treated segment of the skin and its surrounding area to supports determination of location of a treatment energy providing element. Processor 224 could be employed to set upper and lower treated segment of the skin temperature.

Skin treatment based on the thermal image of the treated skin segment allows for adapting a thermal treatment protocol to a particular treated person as well as use of the thermal image for generation of a new thermal treatment protocol to be used in repeat treatments.

Display of the signal being reflective of a thermal image of the treated skin segment and the location of the treatment energy providing element for repositioning of the treatment energy providing element facilitates to receive a homogenous temperature of the treated skin segment.

Use of an infrared camera in combination with a video camera supports treated skin segment temperature visualization and applicator coordinates determination removing some difficult to control variables from the skin treatment process.

What is claimed is:

1. A treatment monitoring system, comprising:
an infrared imager configured to detect infrared radiation from at least one skin segment and to provide a signal reflective of a temperature of the skin segment; and
a processor configured to receive the signal and to determine at least the temperature of at least a portion of the skin segment, wherein the processor is also configured to receive data relating to a location of an applicator configured to apply treatment energy;
wherein the processor is further configured to output a display signal to a display, the display signal being reflective of a thermal image of a treated skin segment and the location of the applicator.

2. The system according to claim 1, wherein the infrared imager has a temperature sensitivity better than 0.50 K.

3. The system according to claim 1, wherein the infrared imager is configured to image an area of the treated skin segment with dimensions of 50×50 mm or smaller.

4. The system according to claim 1, wherein the processor is configured to receive the signal from the infrared imager and update a displayed thermal image of the treated skin segment and the location of the applicator continuously or at predetermined intervals.

5. The system according to claim 1, wherein the processor is configured to generate a skin treatment protocol based of the thermal image of the treated skin segment.

6. The system according to claim 1, wherein a skin treatment energy is provided by one of a group of treatment energy sources consisting of RF energy, ultrasound waves energy, optical radiation energy or a combination thereof.

7. The system according to claim 1, wherein the display signal being reflective of a thermal image of the treated skin segment and the location of the applicator supports repositioning of the applicator such as to receive a uniform/homogenous treated skin segment temperature.

8. The system according to claim 1, wherein a treatment energy sources provide treatment energy sufficient to heat the treated skin segment to a temperature of at least 40 degrees Celsius.

9. A method for visually presenting a thermal image of a treated skin segment, comprising:
using a treatment energy providing element to apply at least one skin treatment energy to a segment of skin and treating by at least one skin treatment energy the segment of skin;
providing an infrared imager configured to capture a thermal image of a treated skin segment and provide a signal reflective of temperature of the treated skin segment; and
communicating the thermal image of the treated skin segment to a processor, the processor configured to process the thermal image and provide the thermal image to a display configured to display a thermal image reflective of the temperature of treated skin segment and location of the treatment energy providing element;
wherein the thermal image supports repositioning of the treatment energy providing element such as to receive a uniform/homogenous treated skin segment temperature.

10. The method according to claim 9, wherein using a thermal image reflective of temperature distribution across the treated segment of the skin supports decision for adaptation or modification of a particular thermal skin treatment.

11. The method according to claim 9, further comprising employing at least one video camera to image the treated segment of the skin and its surrounding area to determine location of a treatment energy providing element.

12. The method according to claim 9, further comprising employing a processor to set upper and lower treated segment of the skin temperature.

13. The method according to claim 9, wherein based on the thermal image adapting a thermal treatment protocol to a particular treated person and employing the thermal image for generation of a new thermal treatment protocol to be used in repeat treatments.

14. The method according to claim 9, wherein using treatment energy sources to provide treatment energy sufficient to heat the treated skin segment to a temperature of at least 40 degrees Celsius.

15. The method according to claim 9, wherein using a display signal being reflective of a thermal image of the treated skin segment and the location of the treatment energy providing element for repositioning of the treatment energy providing element such as to receive a homogenous temperature of the treated skin segment.
